(19) United States
(12) Patent Application Publication
(10) Pub. No.: US 2007/0099887 A1
(43) Pub. Date: May 3, 2007

(54) Photodynamic therapy for the treatment of prostatic conditions

(76) Inventors: Ronald Erwin Boch, North Vancouver (CA); John Robert North, Vancouver (CA); Valery Rubinchik, Richmond (CA); Mitchell D. Visser, Burnaby (CA)

Correspondence Address: MORRISON & FOERSTER LLP, 12531 HIGH BLUFF DRIVE, SUITE 100, SAN DIEGO, CA, 92130-2040, US

(21) Appl. No.: 10/490,470

(22) PCT Filed: Feb. 4, 2004
(86) PCT No.: PCT/CA04/00149

§ 371 Date: Jun. 19, 2006

(30) Foreign Application Priority Data
Feb. 14, 2003 (CA) 2418937
Jul. 30, 2003 (WO) PCT/CA03/01157

(51) Int. Cl. A61K 31/409; A61K 31/555; A61N 1/30

(52) U.S. Cl. 514/185; 514/410; 604/20

(57) ABSTRACT

The present invention relates to a photodynamic method of treating prostatic disorders such as BPH. The method comprises: (i) delivering photosensitizer directly into prostatic tissue of a patient suffering from or suspected of suffering from a prostatic disorder; and (ii) irradiating the prostatic tissue with a light at a wavelength appropriate to activate the photosensitizer.

### FIELD OF THE INVENTION

This invention relates to a method of treating prostatic diseases with photodynamic therapy (PDT). The use of PDT and appropriate photosensitizers for treating prostatic conditions, especially benign prostatic hyperplasia (BPH), is contemplated and disclosed.

### BACKGROUND OF THE INVENTION

The prostate is the organ of the body most often affected by disease in men past middle age. By far the most common condition to affect the prostate is benign prostatic hyperplasia (BPH). Histological evidence of BPH is found in 80% of men over the age of 60. BPH is characterised by a gradual increase in glandular and fibromuscular tissue of the prostate. The proliferation of prostatic tissue associated with BPH can lead to a narrowing or occlusion of the urethra which, in turn, can cause urine outflow obstruction. The disease generally progresses very slowly and symptoms are often mild enough that watchful waiting is the recommended course of action. However, the condition can have serious consequences such as hydronephrosis and renal failure.

There are both medicinal and surgical options for the treatment of BPH. The current therapies are designed to relieve the narrowing of the urethra either through medications or surgery.

Medications include 5-α reductase inhibitors (such as finasteride) and α-adrenoceptor blockers (such as prazosin, terazosin, doxazosin and tamsulosin). Although both classes of drug provide relief of symptomatic BPH the effect wanes over the long-term. In addition, the medications must be taken daily and can have side effects such as dizziness, postural hypotension, ejaculatory dysfunction, decreased libido and impotence.

Surgical therapies for BPH include insterstitial laser coagulation of the prostate (ILC), transurethral microwave thermotherapy (TUMT) which uses microwaves to heat and destroy excess tissue, transurethral needle ablation (TUNA) which uses low-level radiofrequency energy to ablate a defined region of the prostate, and transurethral resection of the prostate (TURP) which is the most commonly used surgical treatment for BPH. TURP involves using an instrument pass through the urethra to remove prostate tissues that surround the urethra. While the current surgical remedies all have reasonable therapeutic outcomes they also have drawbacks. For example, the current therapies are often invasive and can cause damage to tissues other than the target tissue. Also, the surgical procedure can be lengthy, painful, have long recovery periods, necessitate prolonged catheterisation, and require careful monitoring including the use of a rectal probe.

Photodynamic therapy (PDT) has been proposed as one alternative for treating prostatic tissue. U.S. Pat. No. 5,514,669 (Selman) describes a method of treating the symptoms associated with BPH or prostatitis comprising sensitizing the prostatic tissue with an effective amount of photosensitive composition which accumulates in the tissue and exposing the sensitized tissue to a light energy source whereby the photosensitive composition absorbs the light or undergoes a photochemical reaction. The paper entitled “Studies of Tin Ethyl Etiopurpurin Photodynamic Therapy of the Canine Prostate” Journal of Urology, Vol. 165, 1795-1801 (May 2001), Selman et al) described PDT of Canine prostate after a slow bolus intravenous injection of 0.5-11.0 mg/kg of SnET2. Another canine study is detailed in “Photodynamic Therapy in the Canine Prostate Using Motexafm Lutetium” Clinical Cancer Research, Vol. 7, 651-660 (March 2001), His et al. In this study motexafin lutetium was administered to the dogs by intravenous injection.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The present invention relates to a photodynamic method of treating non-cancerous prostatic disorders such as BPH or prostatitis. The method comprises:
(i) delivering photosensitizer directly into prostatic tissue of a patient suffering from or suspected of suffering from a benign prostatic hyperplasia; and
(ii) irradiating the prostatic tissue with a light at a wavelength appropriate to activate the photosensitizer.

In a preferred embodiment, the photosensitizer is delivered directly to the prostate such that the peak concentration of photosensitizer in the prostate is at least 3 mm from the urethral wall.

While not wishing to be bound by theory, it is believed that the use of photodynamic therapy for treating BPH is advantageous because the photosensitizers selectively accumulate in hyperproliferative tissues and, consequently, better target the hyperplastic prostate cells. Also, the fact that both light and drug are required for a photodynamic effect enables more accurate targeting of the hyperplastic tissue. The present method may be used alone or in conjunction with other therapies.

Although the present method has been found useful in non-cancerous prostatic disorders it is believed that the method would have utility for treating cancerous or pre-cancerous prostatic disorders such as prostatic intraepithelial neoplasia (PIN), prostate cancer, and others.

The present method may be used to treat cancerous or pre-cancerous prostatic disorders that are not amenable with surgery. The present method may be used alone or in conjunction with other therapies.

The present method may be used to treat patients who have previously been treated for cancerous or pre-cancerous prostatic disorders but are showing signs of recurrence (for example, through increased PSA levels, worsening prostate biopsies, etc.).

A preferred method of treating cancerous or pre-cancerous prostatic disorders according to the present invention involves:
(i) delivering photosensitizer directly into prostatic tissue of a patient suffering from or suspected of suffering from a cancerous or precancerous prostatic disorder; and
(ii) irradiating the prostatic tissue with a light at a wavelength appropriate to activate the photosensitizer.

In a preferred embodiment, the photosensitizer is delivered directly to the prostate such that the peak concentration of photosensitizer in the prostate is at least 3 mm from the urethral wall.

The present invention also relates to a method of delivering a photosensitizer directly to the prostate such that the peak concentration of photosensitizer in the prostate is at least 3 mm from the urethral wall.

The present invention also relates to a method of delivering a photosensitizer directly to the prostate by injecting photosensitizer into the prostate at least 3 mm from the urethral wall.

The present invention also relates to a device for delivering a photosensitizer directly to the prostate such that the peak concentration of photosensitizer in the prostate is at least 3 mm from the urethral wall and to a device for injecting a photosensitizer into the prostate at least 3 mm from the urethral wall.

As used herein, the term “delivered directly” means non-systemic methods of administering photosensitizer to the target tissue.

### BRIEF DESCRIPTION OF THE DRAWINGS

FIG. 1 shows an activation energy delivery device.

FIG. 2 show an activation energy delivery device in vivo.

### DETAILED DESCRIPTION OF THE INVENTION

The present method involves the photodynamic treatment of prostatic disorders. It is preferred that the present method be used for treating prostatic disorders, especially non-cancerous prostatic disorders. The present method is especially useful in treating BPH.

The method involves the administration of photosensitizer to prostatic tissue of a patient suffering from, or suspected of suffering from, a prostatic disorder. For example, the patient may be suffering from BPH. The photosensitizer is directly delivered to the prostate such that the peak concentration of photosensitizer is at least 3 mm from the wall of the urethra.

Preferably, the photosensitizer herein is directly delivered to the prostate such that the peak concentration of photosensitizer is located at a sufficient distance from the urethra so that diffusion of the photosensitizer towards the urethra results in tissue concentrations immediately adjacent to the urethra or in the urethra itself that are insufficient for adverse PDT reactions. It is preferred that the peak concentration of photosensitizer is at least about 5 mm, more preferably at least about 7 mm, even more preferably at least about 10 mm, away from other tissues such as the bladder neck or urinary sphincter. Furthermore, it is preferred that the peak concentration of photosensitizer is also located at a sufficient distance from the prostatic capsule so that light absorbed by the photosensitizer prevents the light from reaching periprostatic tissues beyond the prostatic capsule.

It has been found that delivering the photosensitizer in such a manner reduces the incidence of photodynamic damage to non-target tissues such as the urethral lumen or healthy prostatic tissue.

It has been found that in certain embodiments of the present method, the photosensitizer diffuses away from the delivery site in a relatively uniform manner which enables the physician to better control the amount of prostatic tissue that is ablated.

It has also been found that, in some embodiments, the present method creates a ‘shadow’ where the high concentration of photosensitizer at the delivery site absorbs much of the activation energy and, consequently, shields the tissue on the far side of the prostate from excessive photodynamic damage.

It has been found that the present method is particularly useful when the patient has a American Urological Association/International Prostate Symptom Score (AUA/IPSS) of greater than 7 (see “Management of BPH”, American Urological Association (2003)).

The photosensitizer must be delivered in such a way as to ensure that the peak concentration of photosensitizer is at least 3 mm from the wall of the urethra. Preferably, the peak concentration of photosensitizer is from 3 mm to about 25 mm from the wall of the urethra. More preferably the peak concentration of photosensitizer is from about 5 mm to about 20 mm from the wall of the urethra. Even more preferably the peak concentration of photosensitizer is from about 7 mm to about 15 mm from the wall of the urethra.

The site of peak concentration of photosensitizer is usually the site of injection. Therefore, it is preferred that the photosensitizer is injected directly into the prostate at least 3 mm from the wall of the urethra. More preferably, the photosensitizer is injected directly into the prostate from 3 mm to about 25 mm from the wall of the urethra. Even more preferably the photosensitizer is injected directly into the prostate from about 5 mm to about 20 mm from the wall of the urethra. Even more preferably still the photosensitizer is injected directly into the prostate from about 7 mm to about 15 mm from the wall of the urethra. It is still more preferred that the photosensitizer is injected directly into the prostate about 10 mm from the wall of the urethra.

It is preferred that the injections are at least about 5 mm, more preferably at least about 7 mm, even more preferably at least about 10 mm away from other tissues such as the bladder neck or urinary sphincter.

Any suitable photosensitizing agent or mixture of agents may be used herein. Generally, these will absorb radiation in the range of from 400 nm to 800 nm, typically from 600 nm to 750 nm.

As used herein, “photosensitizer” or “photosensitizing agent” means a chemical compound which, when contacted by radiation, induces changes to, or destruction of, the prostatic tissue. Preferably, the chemical compound is nontoxic to humans or is capable of being formulated in a nontoxic composition. Preferably, the chemical compound in its photodegraded form is also nontoxic. A listing of photosensitive chemicals may be found in Kreimer-Birnbaum, Sem. Hematol. 26:157-73, 1989 (incorporated herein by reference) and in Redmond and Gamlin, Photochem. Photbiol. 70 (4): 391-475 (1999). The invention may be practiced with a variety of synthetic and naturally occurring photosensitizers, including, but not limited to, pro-drugs such as the pro-porphyrin 5-aminolevulinic acid (ALA) and derivatives thereof, porphyrins and porphyrin derivatives e.g. chlorins, bacteriochlorins, isobacteriochlorins, phthalocyanine and naphthalocyanines and other tetra- and poly-macrocyclic compounds, and related compounds (e.g. pyropheophorbides, sapphyrins and texaphyrins) and metal complexes (such as, but not limited by, tin, aluminum, zinc, lutetium). Tetrahydrochlorins, purpurins, porphycenes, and phenothiaziniums are also within the scope of the invention. Other suitable photosensitizers include bacteriochlorophyll derivatives such as those described in WO-A-97/19081, WO-A-99/45382 and WO-A-01/40232. A preferred bacteriochlorophyll is palladium-bacteriopheophorbide WST09 (Tookad™). Preferably the photosensitizers are selected from pro-porphyrins, porphyrins, and mixtures thereof. Some examples of pro-drugs include aminolevulinic acid such as Levulan™ and aminolevulinic acid esters such as described in WO-A-02/10120 and available as Metvix™, Hexvix™ and Benzvix™. Some examples of di-hydro or tetra-hydro porphyrins are described in EP-A-337,601 or WO-A-01/66550 and available as Foscan™ (temoporfin).

In certain embodiments it is preferred that the photosensitizers are selected from those which photobleach upon exposure to activation energy.

In preferred embodiments of the invention, the photosensitizer is selected from a particularly potent group of photosensitizers known as green porphyrins, which are described in detail in U.S. Pat. No. 5,171,749 (incorporated herein by reference). The term “green porphyrins” refers to porphyrin derivatives obtained by reacting a porphyrin nucleus with an alkyne in a Diels-Alder type reaction to obtain a mono-hydrobenzoporphyrin. Such resultant macropyrrolic compounds are called benzoporphyrin derivatives (BPDs), which is a synthetic chlorin-like porphyrin with various structural analogues, as shown in U.S. Pat. No. 5,171,749. Typically, green porphyrins are selected from a group of tetrapyrrolic porphyrin derivatives obtained by Diels-Alder reactions of acetylene derivatives with protoporphyrin under conditions that promote reaction at only one of the two available conjugated, nonaromatic diene structures present in the protoporphyrin-IX ring systems (rings A and B). Metallated forms of a Gp, in which a metal cation replaces one or two hydrogens in the center of the ring system, may also be used in the practice of the invention. The preparation of the green porphyrin compounds useful in this invention is described in detail in U.S. Pat. No. 5,095,030 (hereby incorporated by reference).

Preferably, the BPD is a benzoporphyrin derivative diester di-acid (BPD-DA), mono-acid ring A (BPD-MA), mono-acid ring B (BPD-MB), or mixtures thereof. These compounds absorb light at about 692 nm wavelength and have improved tissue penetration properties. The compounds of formulas BPD-MA and BPD-MB may be homogeneous, in which only the C ring carbalkoxyethyl or only the D ring carbalkoxyethyl would be hydrolyzed, or may be mixtures of the C and D ring substituent hydrolyzates. A number of other BPD B-ring derivatives may also be used in the present methods. These derivatives have the following general formula:

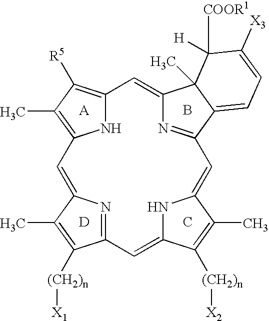

wherein; R⁵ is vinyl, R¹ and R⁶ are methyl, and n is 2. X₁, X₂, and X₃ are listed in the tables below:

TABLE 1
Hydrophilic BPD B-ring analogs

| Drug | X₁ | X₂ | X₃ |
|---|---|---|---|
| QLT0061 | COOH | COOH | COOH |
| QLT0077 | CONH(CH₂)₂N⁺(CH₃)₃I⁻ | CONH(CH₂)₂N⁺(CH₃)₃I⁻ | COOCH₃ |
| QLT0079 | CONH(CH₂)₂N⁺(CH₃)₂((CH₂)₃CH₃ | CONH(CH₂)₂N⁺(CH₃)₂((CH₂)₃CH₃) | COOCH₃ |
| QLT0086 | CONHCH(COOH)CH₂COOH | CONHCH(COOH)CH₂COOH | COOCH₃ |
| QLT0092 | CONH(CH₂)₂NH(CH₃)₂ | CONH(CH₂)₂NH(CH₃)₂ | COOCH₃ |
| | CF₃COO⁻ | CF₃COO− | |
| QLT0094 | CONHCH₂COOH | CONHCH₂COOH | CONHCH₂COOH |

TABLE 2
Lipophilic BPD B-ring analogs

| Drug | X1 | X2 | X3 |
|---|---|---|---|
| QLT0060 | CO(O(CH₂)₂)0H | CO(O(CH₂)₂)0H | COOCH₃ |
| QLT0069 | COOCH₃ | COOCH₃ | COOH |
| QLT0078 | CO(O(CH₂)₂)₂0H | CO(O(CH₂)₂)₂0H | COOCH₃ |
| QLT0080 | CO(O(CH₂)₂)₃OH | CO(O(CH₂)₂)₃OH | COOCH₃ |
| QLT0081 | CO(O(CH₂)₂)₂OCH₃ | CO(O(CH₂)₂)₂OCH₃ | CO(O(CH₂)₂)₂OCH₃ |
| QLT0082 | CO(O(CH₂)₂)₂OH | CO(O(CH₂)₂)₂OH | CO(O(CH₂)₂)₂OH |
| QLT0083 | CO(O(CH₂)₂)₃OH | CO(O(CH₂)₂)₃OH | CO(O(CH₂)₂)₃OH |
| QLT0087 | CO(O(CH₂)₂)₄OH | CO(O(CH₂)₂)₄OH | COOCH₃ |
| QLT0088 | COOCH₃ | COOCH₃ | CONH(C₆H₄)(C₅H₁₀N) |
| QLT0090 | CO(O(CH₂)₂)₅OH | CO(O(CH₂)₂)₅OH | COOCH₃ |
| QLT0093 | CO(O(CH₂)₂)₅OH | CO(O(CH₂)₂)₅OH | CO(O(CH₂)₂)₅OH |

Preferred photosensitizers are the benzoporphyrin derivative mono-acid (BPD-MA), QLT0074 (as set forth in U.S. Pat. No. 5,929,105 referred to therein as A-EA6) and B3 (as set forth in U.S. Pat. No. 5,990,149). Most preferably the photosensitizer is QLT0074 (Lemuteporfin) which has the structure:

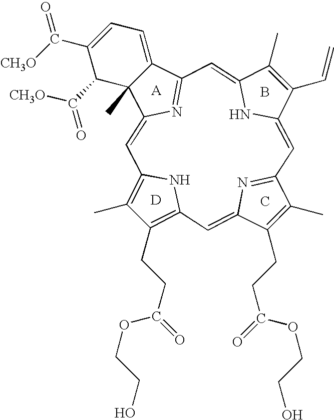

Additionally, the photosensitizers used in the invention may be conjugated to various ligands to facilitate targeting. These ligands include receptor-specific peptides and/orc ligands as well as immunoglobulins and fragments thereof. Preferred ligands include antibodies in general and monoclonal antibodies, as well as immunologically reactive fragments of both.

Dimeric forms of the green porphyrin and dimeric or multimeric forms of green porphyrin/porphyrin combinations can be used. The dimers and oligomeric compounds of the invention can be prepared using reactions analogous to those for dimerization and oligomerization of porphyrins per se. The green porphyrins or green porphyrin/porphyrin linkages can be made directly, or porphyrins may be coupled, followed by a Diels-Alder reaction of either or both terminal porphyrins to convert them to the corresponding green porphyrins. Of course combinations of two or more photosensitizers may be used in the practice of the invention.

In addition to the above mentioned preferred photosensitizing agents, other examples of photosensitizers useful in the invention include, but are not limited to, green porphyrins disclosed in U.S. Pat. Nos. 5,283,255, 4,920,143, 4,883,790, 5,095,030, and 5,171,749; and green porphyrin derivatives, discussed in U.S. Pat. Nos. 5,880,145 and 5,990,149. Several structures of typical green porphyrins are shown in the above cited patents, which also provide details for the production of the compounds.

The photosensitizer can be delivered by any suitable route of administration. For example, transabdominal, transarterial, transrectual, transperineal, or transurethral approaches may be used. Preferably, the photosensitizer is delivered via a transurethral or transrectal approach. More preferably the photosensitizer is delivered via a transurethral approach.

One preferred method of delivering the photosensitizer is by injection. Any suitable injection device may be used. For example, a conventional cytoscope with a needle. Suitable cytoscopes will be familiar to those skilled in the art and include ones available from Karl Storz Endoskope GmbH (Königin-Elisabeth-Str. 60, Berlin, Del.), Olympus America Inc. (2 Corporate Center Drive, Melville, N.Y., USA), ACMI (136 Turnpike Road, Southborough, Mass., USA), Richard Wolf GmbH (Postfach 1164, Knittlingen, Del.).

In order to ensure a good spread of the photosensitizer, it is preferred that the prostatic tissue is injected two or more times, more preferably three or more times, even more preferably four times. It is preferred that the injection(s) are placed so as to avoid the rectum. It is preferred that at least one injection is done in each lobe of the prostate. More preferably two injections are done into each lobe. The injections are preferably placed east-west rather than north-south.

Preferred injection devices include Transurethral Injection Devices (TID). Preferred TID devices are those which can be used in conjunction with conventional cytoscopes. Preferably the TID has a needle suitable for injecting the photosensitizer into the prostatic tissue. For example, the needle can be angled to enable easier injection or it can be flexible enabling the tip to move with the cytoscope. If the needle is angled it preferably has an angle of deflection of more than about 30°, more preferably by more than about 40°, even more preferably by more than about 50°. Especially preferred are needles having an angle of deflection of 60°. The TID) preferably has a 20 g hollow needle. Suitable TID are available from InjecTx™ (San Jose, Calif., USA).

Once the photosensitizer has been delivered to the prostatic tissue it can be activated by any suitable energy source in any suitable manner. It is preferred that the activation energy is delivered directly to the prostate.

Preferably sufficient time is left between delivery of the photosensitizer and administration of the activation energy to allow the photosensitizer to distribute within the target tissue. The exact length of time can vary according to the type of photosensitizer and the target tissue but, in general, it is preferred that at least 5 minutes, more preferably at least 10 minutes, is left between delivery of the photosensitizer and administration of the activation energy.

Preferably, the activation energy comprises a wavelength close to at least one of the absorption peaks of the photosensitizer. This wavelength differs for different photosensitizers. For example, BPD-MA has an absorption peak at 689 nm and so, when BPD-MA is the photosensitizer used, the wavelength of the activation energy is preferably is at or close to 689 nm. The photosensitizer ALA-methyl ester (available under the tradename Metvix) has an absorption peak at 635nm and so when this photosensitizer is used the activation energy is preferable at or close to 635nm. ALA (available under the tradename Levulan) has an absorption peak at 417 nm and at 630 nm so when this photosensitizer is used the activation energy is preferable at or close to 417 nm and/or 630 nm.

Preferable the duration of treatment is short enough so the desired effect is achieved before the drug is washed out of the prostate. It is also preferred that the duration of treatment is short enough to not cause major discomfort to the patient. Preferably, the duration of treatment is less than about 60 mins, more preferably less than about 30 mins, even more preferably less than about 15 mins.

The activation energy should be capable of penetrating the tissue to a depth sufficient to activate the PS at the target tissue. In general, the longer the wavelength of the activation energy, the greater the penetration. Preferably, the activation energy penetrates at least 1 mm, more preferably at least 2 mm, even more preferably at least 3 mm through prostatic tissue.

The activation energy herein may be provided by any suitable means. Generally, the activation energy is provided by a light source although it has been suggested that x-ray or ultrasound sources may be used. Preferred activation energy sources are lasers, filtered full spectrum arc lamps, light emitting diodes, and combinations thereof. More preferably, the energy source used herein is selected from lasers, light emitting diodes, and combinations thereof. Even more preferably the energy source is a laser. Examples of suitable lasers include the 630 PDT (Diomed, Andover, Mass., USA), Ceralas™ (Biolitec AG, Winzerlaer Str.2a, Jena, Del.), and the KTP/532™ or KTP/YAG™ (Laserscope, San Jose, Calif., USA).

The activation energy may be delivered to the prostate by any suitable means. As mentioned above, it is preferred that the activation energy is delivered directly to the prostate. Therefore, it is preferred that the delivery device be adapted or adaptable to deliver activation energy directly to the prostate. Any suitable route of administration can be used. For example, transabdominal, transarterial, transrectual, transperineal, or transurethral may be used. Preferably, the activation energy is delivered via a transurethral or transrectal approach. More preferably the activation energy is delivered via a transurethral approach. Preferred delivery devices are flexible to allow the physician to insert the device through a lumen, especially the urethra, to reach the prostate. Preferably the activation energy is transmitted from the energy source to the target with a fiber optical device. Suitable fiber optics include Optiguide™ Fiber Optics (Diomed, Andover, Mass., USA) and the RD light diffuser (Medlight, Ecublens, CH).

Preferably the activation energy in the present invention is delivered to the prostate by means of a balloon catheter. Some suitable catheters are described in CA-A-2,255,058 (herein incorporated by reference). Preferably, the balloon catheter comprises a treatment window which allows the passage of the activation energy to the prostate. Preferably, the balloon catheter comprises an anchoring balloon. Preferably, the balloon catheter comprises a flexible tip. Preferably, the balloon catheter comprises a light source and a fiber optic. Preferably, the balloon catheter comprises reflective end caps.

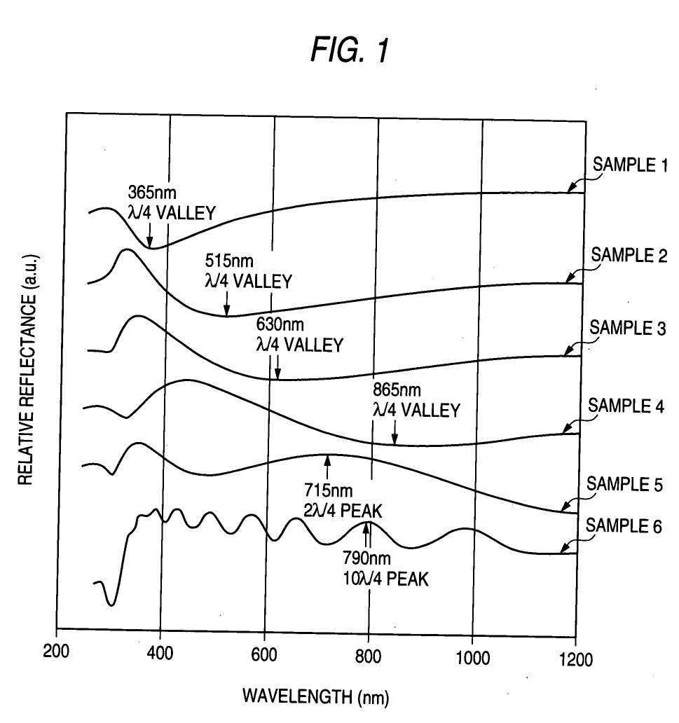

A preferred device for delivery of the activation energy is shown in FIG. 1. The device has a flexible tip (1) and an anchoring balloon (2). There is also a treatment window (3) that allows delivery of the activation energy to the prostate. The device also includes a fiber optic diffuser (4), reflective end-caps (5), and a fiber optic stopper (6).

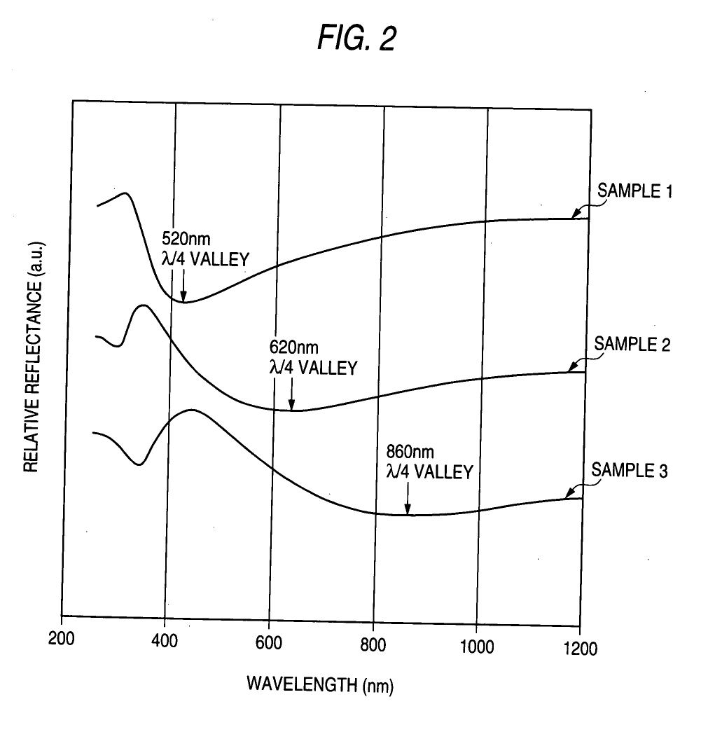

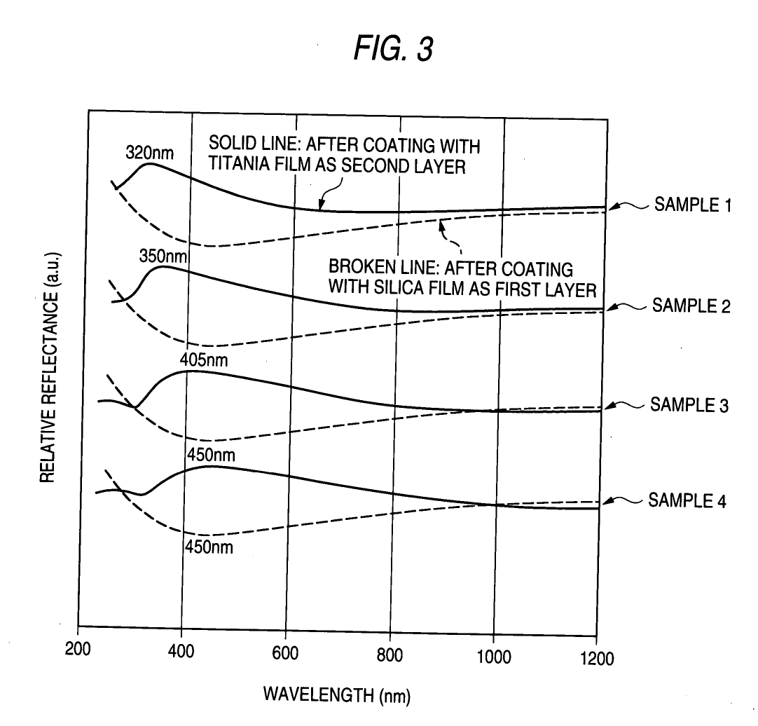

FIG. 2 shows the device of FIG. 1 being used in a transurethral approach. The device (11) is guided via the urethra (10) and the urethral sphincter (9) using the flexible tip (1) until the anchoring balloon (2) is past the bladder neck (6). Once in the bladder (5) the balloon (2) is inflated. In FIG. 2, the distance between the anchoring balloon (2) and the treatment window (3) is approximately 1 cm. This usually puts the treatment window in line with the prostate (7). In this figure, the prostate has been injected (8) twice in each lobe approximately 1 cm from the urethra and the injection sites are also separated by approximately 1 cm. Once the device (11) is in position the activation energy can be delivered via a fiber optic diffuser (4) which is positioned such that the activation energy can exit via the treatment window.

Preferably the activation energy delivery means and the photosensitizer delivery means are combined. This avoids the necessity of inserting two devices and helps ensure that the activation energy is more accurately delivered to the photosensitized target tissue.

A preferred regimen according to the present invention comprises:
a) administering photosensitizer directly to the prostate by transurethral injection. Preferably subjects receive one injection into the prostate either side of the urethra. The preferred injection depth is 10 mm and it is preferred that the injections are at least 10 mm away from other tissues such as the bladder neck or urinary sphincter. The preferred photosensitizer is QLT0074 and the preferred total dose is 0.4 mg.
b) administering activation energy via a transurethral balloon catheter and fiber optic diffuser. Preferably, the activation energy is administered approximately 30 minutes after the injections. The fiber optic diffuser preferably has reflective end caps. If light is the activation energy used it is preferred that a dose between 15 and 200 J/cm² is delivered. More preferred light doses include 25, 20, 80, 120, or 150 J/cm².

### EXAMPLES

It will be understood that the following embodiments of the present invention are intended to be illustrative of some of the possible applications or principles. Various modifications may be made by the skilled person without departing from the true spirit and scope of the invention.

Eight mature, intact, mixed-breed dogs were premedicated with butorphanol (0.4 mg per kilogram of body weight), acepromazine (0.1 mg/kg), and atropine (0.4 mg/kg), all given by intramuscular injection. Anesthesia was induced with intravenous thiopental sodium (8 mg per 0.4536 kilograms of body weight) and maintained with inhaled isoflurane and oxygen (dosage as required for anesthetic effect).

QLT0074 for injection (A-EA6 in U.S. Pat. No. 5,929,105) was reconstituted with Water for Injection to give a stock concentration of 2.0 mg/ml and then diluted with 5% Dextrose in water to a concentration 0.2 mg/ml. Injections of the drug were then delivered to the prostate using a commercially available transurethral injection device (InjecTx™, San Jose, Calif., USA). Two injections of 0.5 ml were given to each left lobe of the prostate and 2 injections of 1.0 ml were given to each right lobe of the prostate.

The activation-energy delivery device used comprised:
a cylindrical fibre optic with a 200 μm diameter core, a 25 mm long diffuser, and a power rating of 175 mW/cm (maximum power load, 437.5 mW),
a reflective-cap fibre-centering balloon catheter with a 20 mm treatment window supplied with a guidewire for positioning the device in the urethra, and
a 3 W 690 nm red-light diode laser (AOC, South Plainfield, N.J., USA).

The treatment assembly was positioned in the urethra using ultrasonic guidance. Ultrasound was also used to position the needles for four percutaneous intraprostatic injections (two per lobe separated by longitudinally by 1 cm). Each injection was at a depth of between 8 and 12 mm into the prostate. The activation energy was delivered 15 minutes after the last injection. A dose of either 25 J/cm², 50 J/cm², or 100 J/cm² was delivered.

The dogs were observed for one week during which time they all appeared to be clinically normal. After seven days the dogs were euthanased and the prostates were harvested. Bowel, bladder, and prostate sizes were found to be within normal ranges. Upon examination, the prostates all exhibited evidence of PDT-associated tissue damage in the target tissue. However, no significant PDT-associated damage was seen in the urethra.

We claim:

1. A photodynamic method of treating prostatic disorders wherein:
i) photosensitizer is administered to prostatic tissue of a patient suffering from or suspected of suffering from a prostatic disorder;
ii) the prostatic tissue of said patient is irradiated with a light energy at a wavelength appropriate to activate the photosensitizer;
wherein the photosensitizer is delivered directly into the prostatic tissue such that the peak concentration of photosensitizer in the prostate is at least 3 mm from the urethral wall.

2. A photodynamic method of treating non-cancerous prostatic disorders wherein:
i) photosensitizer is administered to prostatic tissue of a patient suffering from or suspected of suffering from a prostatic disorder;
ii) the prostatic tissue of said patient is irradiated with a light energy at a wavelength appropriate to activate the photosensitizer;
wherein the photosensitizer is delivered directly into the prostatic tissue such that the peak concentration of photosensitizer in the prostate is at least 3 mm from the urethral wall.

3. A method according to claim 1 wherein the prostatic disorder is benign prostatic hyperplasia.

4. A method according to claim 1 wherein the peak concentration of photosensitizer is from 3 mm to about 25 mm from the wall of the urethra.

5. A method according to claim 1 wherein the peak concentration of photosensitizer is from about 5 mm to about 20 mm from the wall of the urethra.

6. A method according to claim 1 wherein the peak concentration of photosensitizer is from about 7 mm to about 15 mm from the wall of the urethra

7. A method according to claim 1 wherein the photosensitizer is delivered by transurethral injection into the prostate.

8. A method according to claim 1 wherein the photosensitizer is selected from pro-porphyrins, porphyrins, and mixtures thereof.

9. A method according to claim 1 wherein the photosensitizer is selected from green porphyrins.

10. A method according to claim 1 wherein the activation energy is delivered by means of a lasers, fibre optical illumination devices, or combinations thereof.

11. A method according to claim 1 wherein the activation energy is delivered transurethrally.

12. A photodynamic method of treating prostatic disorders wherein:
i) photosensitizer is administered to prostatic tissue of a patient suffering from or suspected of suffering from a prostatic disorder;
ii) the prostatic tissue is irradiated with a light at a wavelength appropriate to activate the photosensitizer;
wherein the photosensitizer is injected into the prostatic tissue at least 3 mm from the urethral wall.

13. A method according to claim 12 wherein the prostatic disorder is benign prostatic hyperplasia.

14. A method according to claim 12 wherein the photosensitizer is injected from 3 mm to about 25 mm from the wall of the urethra.

15. A method according to claim 12 wherein the photosensitizer is injected from about 5 mm to about 20 mm from the wall of the urethra.

16. A method according to claim 12 wherein the photosensitizer is delivered by transurethral injection into the prostate.

17. A method according to claim 12 wherein the photosensitizer is selected from pro-porphyrins, porphyrins, and mixtures thereof.

18. A method according to claim 12 wherein the activation energy is delivered transurethrally.

19. A photodynamic method of treating benign prostatic hyperplasia wherein:
(i) photosensitizer is directly delivered to the prostate, and
(ii) the prostatic tissue is irradiated with a light at a wavelength appropriate to activate the photosensitizer;
wherein the photosensitizer is administered such that the peak concentration in the prostate is a sufficient distance from the urethra so that diffusion of the photosensitizer towards the urethra results in tissue concentrations immediately adjacent to the urethra that are insufficient to cause an adverse photodynamic reaction.

20. A photodynamic method of treating benign prostatic hyperplasia wherein:
(i) photosensitizer is directly delivered to the prostate, and
(ii) the prostatic tissue is irradiated with a light at a wavelength appropriate to activate the photosensitizer;
wherein the peak concentration of photosensitizer in the prostate is a sufficient distance from the prostatic capsule so that light absorbed by the photosensitizer prevents the light from reaching periprostatic tissues beyond the prostatic capsule.
